(19) United States
(12) Patent Application Publication
(10) Pub. No.: US 2026/0216145 A1
(43) Pub. Date: Jul. 30, 2026

(54) COMPOSITION AND PRODUCTS COMPRISING AN ANTIBIOTIC AND A GLUCOCORTICOID FOR TREATING MASTITIS

(71) Applicant: VIRBAC, CARROS (FR)

(72) Inventors: Matthew WELLS, Hamilton (NZ); Nicola CUFF, Auckland (NZ)

(21) Appl. No.: 19/142,429

(22) PCT Filed: Dec. 20, 2023
(86) PCT No.: PCT/EP2023/087037

§ 371 Date: Jun. 23, 2025

(30) Foreign Application Priority Data
Dec. 23, 2022 (EP) 22307019.4

(51) Int. Cl. A61K 31/43; A61K 31/245; A61K 31/573; A61P 31/04

(52) U.S. Cl. CPC A61K 31/43; A61K 31/245; A61K 31/573; A61P 31/04

(57) ABSTRACT

The present disclosure mainly relates to a composition comprising, in a pharmaceutically acceptable medium, (i) penicillin G or a salt thereof and (ii) hydrocortisone aceponate. It also relates to a composition comprising, in a pharmaceutically acceptable medium, (i) at least one antibiotic chosen from penicillin, its derivatives and its salts, and (ii) at least one glucocorticoid, for use for treating or the prophylaxis of bacterial infectious diseases in lactating ruminants, in particular for treating mastitis. It also relates to veterinary pharmaceutical products containing (i) an antibiotic chosen from penicillin, its derivatives and its salts, and (ii) a glucocorticoid, as a combined preparation for simultaneous, separate or sequential use for treating bacterial infectious diseases in lactating ruminants, in particular for treating mastitis. It also relates to such compositions or such products for use in the treatment or the prophylaxis of bacterial infectious diseases in lactating ruminants, in particular mastitis in lactating animals producing milk for human consumption. Finally, it also relates to such compositions or such products for uses in improving the health of an infected lactating ruminants.

The present disclosure mainly relates to a composition comprising, in a pharmaceutically acceptable medium, (i) at least one antibiotic chosen from penicillin, its derivatives and its salts, and (ii) at least one glucocorticoid, for use for treating bacterial infectious diseases in lactating ruminants, in particular for treating mastitis. It also relates to veterinary pharmaceutical products containing (i) an antibiotic chosen from penicillin, its derivatives and its salts, and (ii) a glucocorticoid, as a combined preparation for simultaneous, separate or sequential use for treating bacterial infectious diseases in lactating ruminants, in particular for treating mastitis. It also relates to the use of such compositions or such products for the treatment or the prophylaxis of bacterial infectious diseases in lactating ruminants, in particular mastitis in lactating animals producing milk for human consumption. The present disclosure also relates to a composition comprising, in a pharmaceutically acceptable medium, (i) at least one antibiotic chosen from penicillin G procaine, its derivatives and its salts, and (ii) at least one glucocorticoid, preferentially hydrocortisone aceponate (HCA). Finally, it also relates to such compositions or such products for uses in improving the health of an infected lactating ruminants.

Mastitis is a common epithelial microbial infection, especially in domestic animals raised for obtaining milk such as a dairy cow and goat. Mastitis is a disease in which the mammary glands in the animals are infected with Staphylococcus (e.g. Staphylococcus aureus), Streptococcus, Escherichia coli or another pathogenic bacterium causing inflammation; or a disease related to such a bacterium. Disease may range from subclinical (i.e. presence of bacteria and elevated somatic cell count (SCC) but with no grossly evident changes in milk, mammary gland tissue or the cow) to clinical (i.e. heat and swelling of the mammary gland, grossly evident changes to the milk including the presence of clots or flecks). In severe cases, systemic signs of illness may appear such as depression and dehydration and occasionally death.

Mastitis is a very costly disease for the dairy industry due to its high incidence, and the resulting reduced milk production, reduced milk quality and increased culling (removal) of animals from dairy herds. Mastitis is thus a very serious disease in domestic animals, and a known therapy frequently used is the administration by infusion of an antimicrobial drug directly into the mammary gland of a domestic animal affected with mastitis. Most of the mastitis is cured or alleviated by such an infusion.

However, bacteria causing mastitis are becoming less susceptible to existing mastitis antimicrobial drugs.

Among the available treatments, the use of antibiotics such as penicillin is well-established to treat mastitis during lactation.

However, milk collected from animals within a few days of this treatment and intended for human consumption must be discarded (i.e. withholding period). Accordingly, there is a commercial disadvantage to farmers if milk or colostrum collected from treated cows cannot be sold.

The withholding period (WHP), also called withdrawal period, is the minimum period of time that must elapse between (i) the last administration or application of a veterinary chemical, including treated feed, to an animal, and (ii) slaughter, collection, harvesting and/or use of animal foodstuffs for human consumption. It is a reflection of the chemical residue levels in the treated animal and its by-products. The objective of a WHP is to provide users with the information they need to ensure that residues in foodstuffs for human consumption will not exceed the maximum residue limit (MRL).

It is desirable to reduce the chemical residue levels in livestock and in animals products used for human consumption, such as cattle treated with an antibiotic based composition, so that consumer safety is increased. Moreover, it is economically important that the withholding periods for ruminants are also reduced so that cattle owners are able to market their livestock and milk quicker after treatment and stop the watching over the treated animals. By reducing the withholding periods, the owners also reduce the time period where the milk is discarded and thus reduce waste.

There is thus a need for an improved and efficient therapy of mastitis in non-human mammals, which may provide additional benefits, such as reduced withholding periods. Indeed, there is a need to improve milk production, improve milk quality and/or decrease culling of animals from dairy herds. Especially, in infected lactating ruminants there is a need for an efficient therapy of mastitis in lactating animals, where a short withholding period (WHP) is desired.

The present invention fulfills these needs.

The inventors have discovered that a combined treatment of a penicillin such as benzylpenicillin (especially penicillin G procaine), with a glucocorticoid such as hydrocortisone aceponate (HCA), is able to effectively treat mastitis in non-human mammals, preferably lactating ruminants such as cows. Surprisingly, as shown in the examples, such combinations are furthermore able to reduce significantly the WHP. Indeed, as demonstrated, the WHP is reduced by at least several hours and up to 24 h compared to existing treatment. This benefit is of the utmost importance, especially economically, as it allows gaining time of milk production, reducing milk waste and stopping watching over the treated animals.

It is hypothesized that the glucocorticoid component in the veterinary composition, such as hydrocortisone aceponate (HCA), impacts the bioavailability and pharmacokinetics of the penicillin component, such as benzylpenicillin, so that the WHP for the antibiotic is reduced (and thus improved) for human consumption.

The present disclosure relates to a veterinary composition comprising, in a pharmaceutically acceptable medium, (i) at least one antibiotic chosen from penicillin, its derivatives and its salts, and (ii) at least one glucocorticoid (such as hydrocortisone aceponate (HCA)), for use for reducing the withholding period of the antibiotic compared to the same veterinary composition without the glucocorticoid. Said veterinary composition may comprise at least two antibiotics, wherein at least one antibiotic is chosen from penicillin, its derivatives and its salts, and at least one other is chosen from beta-lactam antibiotics.

The present invention also relates to a composition comprising, in a pharmaceutically acceptable medium, (i) at least one antibiotic chosen from penicillin, its derivatives and its salts, and (ii) at least one glucocorticoid (such as hydrocortisone aceponate (HCA)), for use for the treatment or the prophylaxis of bacterial infectious diseases in infected lactating ruminants, in particular mastitis, in infected lactating ruminants producing milk for human consumption. Preferentially, penicillin and its derivatives are chosen from compounds of formula (I):

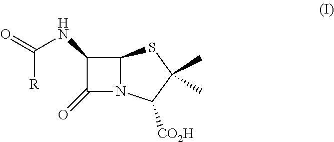

wherein R is an aralkyl, a phenoxymethyl radical, an alkyl radical having 1 to 10 carbon atoms or an alkenyl radical having 2 to 10 carbon atoms.

Preferably, said composition does not comprise any zirconium containing compound.

Said composition may comprise at least two antibiotics, wherein at least one antibiotic is chosen from penicillin, its derivatives and its salts, and at least one other is chosen from beta-lactam antibiotics.

The disclosure also relates to such compositions for uses to improve the health of an infected lactating ruminant, in particular mastitis in an infected lactating ruminants producing milk for human consumption.

The present invention also relates to compositions containing (i) an antibiotic chosen from penicillin, its derivatives and its salts, and (ii) a glucocorticoid (such as hydrocortisone aceponate (HCA)), as a combined preparation for simultaneous, separate or sequential use for treating bacterial infectious diseases in infected lactating ruminants, in particular for treating mastitis. Said compositions of the invention are also called “kit-of-parts” or “products of the disclosure” in the present application. For a separate or sequential use, the compositions are constituted of two separate products, one comprising (i) an antibiotic chosen from penicillin, its derivatives and its salts and one comprising (ii) a glucocorticoid (such as hydrocortisone aceponate (HCA)).

In particular, the composition according to the invention does not comprise any zirconium-containing compound, i.e. the composition is devoid of any zirconium-containing compound. In the present application, by “zirconium-containing compound”, it is meant any compound comprising a zirconium atom (Zr). The zirconium-containing compound may be chosen from zirconium salts, especially organic and inorganic salts of zirconium. Among the organic salts of zirconium, one can quote the salts of zirconium and of carboxylic acids, such as zirconium stearate, zirconium acetate or sodium zirconium lactate. Preferably the composition according to the invention does not comprise any zirconium-containing compound chosen from zirconium salts, preferably chosen from organic and inorganic salts of zirconium, preferably chosen from zirconium stearate, zirconium acetate and sodium zirconium lactate.

By “simultaneous use”, it is meant that each composition (i.e. the one containing (i) an antibiotic chosen from penicillin, its derivatives and its salts, and the one containing (ii) a glucocorticoid, and (iii) optionally a second antibiotic chosen from beta-lactam antibiotics) is applied according to the same route and at the same time or sensibly the same time. By “separate use”, it is meant that each composition (i.e. the one containing (i) an antibiotic chosen from penicillin, its derivatives and its salts, and the one containing (ii) a glucocorticoid, and (iii) optionally a second antibiotic chosen from beta-lactam antibiotics) is applied according to different routes and at the same time or sensibly the same time. By “sequential” use, it is meant that each composition (i.e. the one containing (i) an antibiotic chosen from penicillin, its derivatives and its salts, and the one containing (ii) a glucocorticoid, and (iii) optionally a second antibiotic chosen from beta-lactam antibiotics) is applied at different times, the route of administration being identical or different (preferably identical). Preferably the sequential use is a successive administration.

Preferably, the compositions of the invention containing (i) an antibiotic chosen from penicillin, its derivatives and its salts, and (ii) a glucocorticoid, are used as a combined preparation for simultaneous or sequential use for treating mastitis.

The present invention thus relates to a composition comprising, in a pharmaceutically acceptable medium, (i) benzylpenicillin (penicillin G) or a salt thereof as a first active ingredient (preferably penicillin G procaine), and (ii) hydrocortisone aceponate (HCA) as a second active ingredient.

The present invention also relates to a composition comprising, in a pharmaceutically acceptable medium, (i) benzylpenicillin (penicillin G) or a salt thereof as a first active ingredient (preferably penicillin G procaine), and (ii) hydrocortisone aceponate (HCA) as a second active ingredient, for use as a medicament.

In a preferred embodiment, the composition is for use for treating or for the prophylaxis of bacterial infectious diseases in infected lactating ruminants, preferably for use for treating mastitis.

In one embodiment of the invention, the first active ingredient (i) is penicillin G procaine.

By “bacterial infectious disease”, it is meant any infectious disease due to bacteria. Preferably, said bacteria are Gram-positive, preferably chosen from Streptococcal species and Staphylococcal species. Streptococcus uberis and Staphylococcus aureus are examples of Gram-positive Streptococcal species. Coagulase-negative Staphylococci (CNS) is a subgroup of Gram-positive Staphylococcal species. The CNS typically include Staphylococcus xylosus, Staphylococcus sciuri and/or Staphylococcus saprophyticus. Preferably, the bacterial infectious disease is mastitis.

In a preferred embodiment, the composition is for use against Gram-positive mastitis organisms, including but not limited to:
Streptococcal species such as Streptococcus uberis, and/or
Staphylococcal species including coagulase-negative Staphylococci such as Staphylococcus xylosus, Staphylococcus sciuri and/or Staphylococcus saprophyticus, and penicillin-sensitive Staphylococcus aureus.

In another preferred embodiment, the composition is also for use for the reduction of the clinical signs of udder inflammation such as heat, swelling, clots and elevated somatic cell count (SCC) associated with mastitis.

The present invention also relates to a composition comprising, in a pharmaceutically acceptable medium, (i) penicillin G or a salt thereof as a first active ingredient (preferably penicillin G procaine), and (ii) hydrocortisone aceponate as a second active ingredient, for use in treating or for the prophylaxis of bacterial infectious diseases in infected lactating ruminants, preferably for use for treating mastitis, wherein the withholding period (WHP) of penicillin G or a salt thereof in the lactating ruminant tissues and/or by-products is reduced. Preferably the by-product is milk and/or the tissue is meat.

By the feature “the withholding period of penicillin G or a salt thereof in the lactating ruminant tissues and/or by-products is reduced”, it is meant that the WHP of penicillin G or a salt thereof in the tissues and/or by-products of an infected lactating ruminant is reduced when administered together with hydrocortisone aceponate, as compared to the WHP of penicillin G in the tissues and/or by-products when said penicillin G or a salt thereof is administrated alone (i.e. without any glucocorticoid) to the infected lactating ruminant.

In another embodiment, the present invention also relates to a process for collecting milk from a lactating ruminant, which comprises:
a) measuring the period that starts upon the most recent administration of a composition comprising, in a pharmaceutically acceptable medium, (i) penicillin G or a salt thereof, preferably penicillin G procaine, and (ii) hydrocortisone aceponate, to said lactating ruminant; and
b) collecting milk from said lactating ruminant,
   wherein step (b) is performed when the measured period of step (a) lies between 92 h and 98 h in the case of once-a-day milking ruminant,
   and step (b) is performed when the measured period of step (a) lies between 70 h and 86 h in the case of twice-a-day milking ruminant.

In another embodiment, the present invention also relates to a method of treating a lactating ruminant, which comprises:
a) administrating a composition according to the invention to said lactating ruminant. Preferably the composition comprises, in a pharmaceutically acceptable medium, penicillin G or a salt thereof, preferably penicillin G procaine, and a glucocorticoid, preferably hydrocortisone aceponate;
b) measuring the period that starts upon the most recent administration of said composition to said lactating ruminant; and
c) collecting milk from said lactating ruminant,
   wherein step (c) is performed when the measured period of step (b) lies between 92 h and 98 h in the case of once-a-day milking ruminant,
   and step (c) is performed when the measured period of step (b) lies between 70 h and 86 h in the case of twice-a-day milking ruminant.

In another embodiment, the present invention relates to a method for lowering somatic cell count in the milk of a lactating ruminant infected with a mastitis-causing organism, which comprises a step of administering to said infected lactating ruminant a composition comprising, in a pharmaceutically acceptable medium, (i) at least one antibiotic chosen from penicillin, its derivatives and its salts, and (ii) at least one glucocorticoid, preferably HCA. By “lowering somatic cell count (SCC)” in the milk, it is meant lowering SCC as compared to the SCC in the milk when said antibiotic is administrated alone (i.e. without any glucocorticoid) to the infected lactating ruminant.

In another embodiment, the present invention relates to a composition for use in lowering somatic cell count in the milk of a lactating ruminant infected with a mastitis-causing organism, which comprises in a pharmaceutically acceptable medium, (i) at least one antibiotic chosen from penicillin, its derivatives and its salts, and (ii) at least one glucocorticoid, preferably HCA.

The present invention also relates to a kit comprising:
a) a composition comprising, in a pharmaceutically acceptable medium, (i) benzylpenicillin (penicillin G) or a salt thereof as a first active ingredient (preferably penicillin G procaine), and (ii) hydrocortisone aceponate (HCA) as a second active ingredient,
b) a notice of use of the composition, and
optionally c) an administration device, preferably a intramammary syringe.

In an alternative more preferred embodiment, the composition comprises, in a pharmaceutically acceptable medium, a first active ingredient (i) that is at least one antibiotic chosen from penicillin, its derivatives and its salts, and a second active ingredient (ii) that is at least one glucocorticoid, preferably HCA. In such embodiment, the invention relates to a veterinary composition comprising, in a pharmaceutically acceptable medium, (i) at least one antibiotic chosen from penicillin, its derivatives and its salts, and (ii) at least one glucocorticoid, preferably HCA, for use in the treatment or the prophylaxis of bacterial infectious diseases in infected lactating ruminants. Said composition may comprise at least two antibiotics as first active ingredients (i), wherein at least one antibiotic is chosen from penicillin, its derivatives and its salts, and at least one other is chosen from beta-lactam antibiotics.

In a preferred embodiment, the at least one antibiotic chosen from penicillin, its derivatives and its salts is penicillin G and more preferably penicillin G procaine, and the at least one glucocorticoid is hydrocortisone aceponate. Preferably, penicillin and its derivatives are chosen from compounds of formula (I):

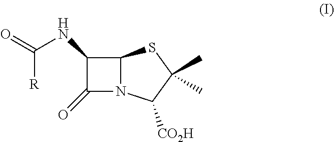

wherein R is an aralkyl, a phenoxymethyl radical, an alkyl radical having 1 to 10 carbon atoms or an alkenyl radical having 2 to 10 carbon atoms.

Preferably said composition does not comprise any zirconium containing compound.

The lactating ruminant according to the invention is advantageously selected from the group consisting of dairy cattle, milking goats, beef cattle, sheep, deer, milking buffalo and yaks. Preferably, the lactating ruminants are dairy cattle.

In a preferred embodiment the bacterial infectious disease is mastitis in lactating ruminants producing milk for human consumption. In another preferred embodiment the lactating ruminant is cattle. Especially preferably the lactating ruminant is cow.

The present disclosure also relates to the use of a composition comprising, in a pharmaceutically acceptable medium, at least one glucocorticoid (preferably HCA), for treating mastitis in a non-human mammal treated by at least one antibiotic chosen from penicillin, its derivatives and its salts.

The non-human mammal is preferably a lactating ruminant.

The use according to the present disclosure preferably allows the treatment of mastitis in a lactating ruminant. In a preferred embodiment, the use according to the present invention also allows the treatment of mild mastitis in a lactating ruminant.

It has surprisingly been found that the use of such composition with at least one antibiotic chosen from penicillin, its derivatives and its salts and at least one glucocorticoid (preferably HCA) remarkably reduced the withholding period compared to a prior art composition comprising the antibiotic alone.

In yet another preferred embodiment, the present invention relates also to the use of a composition comprising, in a pharmaceutically acceptable medium, (i) at least one antibiotic chosen from penicillin, its derivatives and its salts, and (ii) at least one glucocorticoid (preferably HCA), for the treatment or the prophylaxis of bacterial infectious diseases in infected lactating ruminants, preferably wherein the withholding period (WHP) of the antibiotic in the milk of said ruminants is reduced. By the feature “the withholding period of the antibiotic in the milk of said ruminants is reduced”, it is meant that the WHP of the antibiotic in the milk of an infected lactating ruminant is reduced as compared to the WHP of the same antibiotic in the milk when said antibiotic is administrated alone (i.e. without any glucocorticoid) to the infected lactating ruminant.

In a preferred embodiment, the at least one antibiotic chosen from penicillin, its derivatives and its salts is penicillin G and more preferably penicillin G procaine, and the at least one glucocorticoid is hydrocortisone aceponate.

In another preferred embodiment, the composition is also for use for the reduction of the clinical signs of udder inflammation such as heat, swelling, clots and elevated somatic cell count (SCC) associated with mastitis.

In an alternative more preferred embodiment, the lactating ruminant are producing milk for human consumption and the withholding period is reduced for the milk consumption by humans.

In another alternative preferred embodiment, the present invention also relates to the use of a composition comprising, in a pharmaceutically acceptable medium, at least one glucocorticoid (preferably HCA), for enhancing the removal of an antibiotic chosen from penicillin, its derivatives and its salts (preferably penicillin G and more preferably penicillin G procaine), in an infected lactating ruminant treated by said antibiotic. Preferably, the present invention relates to the use of a composition comprising, in a pharmaceutically acceptable medium, hydrocortisone aceponate, for enhancing the removal of an antibiotic chosen from penicillin, its derivatives and its salts (preferably penicillin G and more preferably penicillin G procaine), in an infected lactating ruminant treated by said antibiotic. In particular, penicillin and its derivatives are chosen from compounds of formula (I):

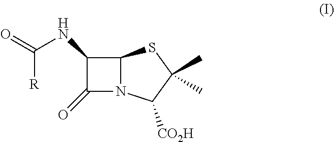

wherein R is an aralkyl, a phenoxymethyl radical, an alkyl radical having 1 to 10 carbon atoms or an alkenyl radical having 2 to 10 carbon atoms.

Preferably said composition does not comprise zirconium containing compounds.

Preferably, said composition comprises at least one antibiotic chosen from penicillin, its derivatives and its salts that is preferably penicillin G and more preferably is penicillin G procaine, and at least one glucocorticoid, that is preferably hydrocortisone aceponate. Alternatively, said composition does not contain one antibiotic chosen from penicillin, its derivatives and its salts.

By “enhancing the removal of an antibiotic chosen from penicillin, its derivatives and its salts”, it is meant that the antibiotic residue in an infected lactating ruminant treated by said antibiotic and by at least one glucocorticoid (preferably HCA) is below maximum residue limit (MRL) in a significant shorter time than when treated by said antibiotic alone. Preferably, the significant shorter time is at least 6 h, preferably at least 12 h, preferably at least 18 h, more preferably at least 24 h. Preferably “enhancing the removal of an antibiotic chosen from penicillin, its derivatives and its salts” means that the antibiotic residue in an infected lactating ruminant treated by said antibiotic and by hydrocortisone aceponate is below maximum residue limit within at least 6 h, preferably at least 12 h, preferably at least 18 h, more preferably at least 24 h.

Preferably, the addition of the glucocorticoid enhances the removal of the antibiotic from the udder of the lactating ruminant.

In yet another preferred embodiment, the present invention also relates to a method for lowering somatic cell count in the milk of a lactating ruminant infected with a mastitis-causing organism, which comprises a step of administering to said infected lactating ruminant a composition comprising, in a pharmaceutically acceptable medium, (i) at least one antibiotic chosen from penicillin, its derivatives and its salts, and (ii) at least one glucocorticoid. In a preferred embodiment, the at least one antibiotic chosen from penicillin, its derivatives and its salts is penicillin G, and more preferably penicillin G procaine, and the at least one glucocorticoid is hydrocortisone aceponate. In particular, penicillin and its derivatives are chosen from compounds of formula (I):

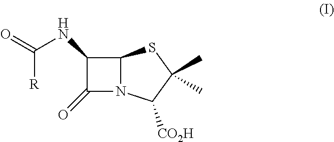

wherein R is an aralkyl, a phenoxymethyl radical, an alkyl radical having 1 to 10 carbon atoms or an alkenyl radical having 2 to 10 carbon atoms.

The terms “treat”, “treating” and “treatment”, as used herein, refer to any action providing a benefit to a subject afflicted with a condition or disease (preferably a non-human mammal afflicted by mastitis and more preferably an infected lactating ruminant), including improvement in the condition through lessening or suppression of at least one symptom, and/or delay in progression of the disease, and/or prevention or delay in the onset of the disease. The treatment of mastitis includes a reduction of the clinical symptoms of mastitis and/or an inhibition of the microbial overgrowth/multiplication of pathogenic bacteria, yeasts and/or fungi, involved in mastitis.

The words “comprise” or “comprises” or “comprising” are to be understood as to imply the inclusion of a stated item or group of items, but not the exclusion of any other item or group of items.

The words “consist” or “consists” or “consisting” are however used to mean the inclusion of a stated item or group of items, and the exclusion of any other item or group of items.

### Antibiotic

The present invention requires (i) at least one antibiotic chosen from penicillin, its derivatives and its salts.

Penicillin, its derivatives and its salts (penams) belong to beta-lactam antibiotics.

Penicillin and its derivatives may be natural of semi-synthetic. They are of formula (I):

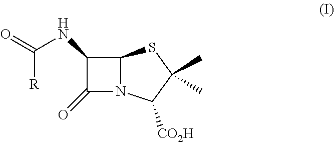

wherein R is an aralkyl, a phenoxymethyl radical, an alkyl or an alkenyl radical.

By “alkyl radical”, it is meant a linear or branched hydrocarbon chain, preferably having 1 to 10 carbon atoms, preferably 1 to 8. Preferably, the alkyl radical is methyl, ethyl, propyl, n-butyl, isopropyl, n-pentyl, n-hexyl or n-heptyl. Preferably, the alkyl radical is methyl or n-heptyl.

By “aralkyl”, it is meant an aryl, substituted or not, linked to an alkyl radical. The aryl is preferably phenyl or p-hydroxyphenyl. Preferably, the aralkyl is benzyl or p-hydroxybenzyl.

By “alkenyl radical”, it is meant meant a linear or branched hydrocarbon chain, preferably having 2 to 10 carbon atoms, preferably 2 to 8, and comprising one unsaturated bond. Preferably, the alkenyl radical is ethynyl, propynyl, butynyl, pentenyl or hexenyl. Preferably, the alkenyl radical is 2-pentenyl.

By “salt”, it is meant a salt of penicillin or a derivative thereof with an alkaline or alkaline-earth cation. Preferably, the salt is a sodium or potassium salt.

Preferably, the compound of formula (I) is such that R is an aralkyl, more preferably benzylpenicillin (penicillin G) or p-hydroxybenzylpenicillin (penicillin III or X).

Preferably, the compound of formula (I) is such that R is a phenoxymethyl radical, more preferably phenoxymethylpenicillin (penicillin V).

Preferably the compound of formula (I) is such that R is an alkyl, preferably n-heptyl, more preferably the compound of formula (I) is n-heptylpenicillin (penicillin IV or K).

Preferably the compound of formula (I) is such that R is an alkenyl, preferably 2-pentenyl, more preferably the compound of formula (I) is 2-pentenylpenicillin (penicillin I or F).

Thus, preferably, the antibiotic is chosen from penicillin G (benzylpenicillin), p-hydroxybenzylpenicillin, phenoxymethylpenicillin, n-heptylpenicillin and 2-pentenylpenicillin.

More preferably, the antibiotic is benzylpenicillin (penicillin G).

Penicillin G exerts a bactericidal action against penicillin-susceptible microorganisms during the stage of active multiplication. It acts through the inhibition of biosynthesis of cell-wall peptidoglycan, rendering the cell wall osmotically unstable.

Procaine benzylpenicillin (INN), also known as procaine G penicillin or penicillin G procain, is an injectable antibiotic. It is a poorly soluble salt form of penicillin which is a combination of naturally occurring benzylpenicillin (penicillin G) and the local anesthetic agent procaine in equimolar amounts.

More preferably, the antibiotic is penicillin G procaine.

The composition of the invention may comprise at least two antibiotics, wherein at least one antibiotic is chosen from penicillin, its derivatives and its salts and at least one other antibiotic is chosen from beta-lactam antibiotics. By “beta-lactam antibiotics”, it is meant antibiotics chosen from penicillin derivatives (penams), cephalosporins and cephamycins (cephems), monobactams, carbapenems and carbacephems. Preferably, the beta-lactam antibiotic is a penicillin derivative or a cephalosporin. More preferably, the beta-lactam antibiotic is chosen from cloxacillin, oxacillin, amoxicillin, ampicillin, nafcillin and methicillin, preferably is cloxacillin.

### Glucocorticoid

The present invention further requires (ii) at least one glucocorticoid, preferably HCA.

Glucocorticoids (also called steroids, corticosteroids or cortisone analogues) are compounds that are generally used for local application (such as nasal, cutaneous or ophthalmic). Said glucocorticoid may be a monoester or a diester. Preferably the glucocorticoid of the invention is a diester.

The glucocorticoid according to the invention may be selected in the group comprising alclometasone dipropionate, alclometasone propionate, amcinonide, beclomethasone dipropionate, betamethasone acetate, betamethasone dipropionate, betamethasone sodium phosphate, betamethasone benzoate, betamethasone valerate, budesonide, clobetasol propionate, clobetasol butyrate, clocortolone pivalate, desonide, dexamethasone acetate, dexamethasone nicotinate, dexamethasone propionate, dexamethasone valerate, dexamethasone sodium phosphate, desoximetasone, diflorasone diacetate, diflucortolone valerate, halobetasol propionate, flumethasone pivalate, fluocinolone acetonide, fluocinonide, flurandrenolide, fluticasone propionate, halcinonide, halobethasone propionate, halomethasone, a hydrocortisone such as benzodrocortisone (hydrocortisone 17-benzoate), hydrocortamate (hydrocortisone 21-(diethylamino) acetate), hydrocortisone aceponate (hydrocortisone 21-acetate 17α-propionate or HCA), hydrocortisone acetate, hydrocortisone bendazac, hydrocortisone buteprate (hydrocortisone 17a-butyrate 21-propionate), hydrocortisone butyrate (hydrocortisone 17a-butyrate), hydrocortisone 21-butyrate, hydrocortisone cypionate (hydrocortisone cyclopentanepropionate), hydrocortisone hydrogen succinate, hydrocortisone phosphate, hydrocortisone sodium phosphate, hydrocortisone sodium succinate, hydrocortisone succinate (hydrocortisone hemisuccinate), hydrocortisone tebutate, hydrocortisone valerate, hydrocortisone xanthogenic acid and hydrocortisone probutate; methylprednisolone acetate, methylprednisolone sodium succinate, methylprednisolone aceponate, mometasone furoate, prednisolone sodium phosphate, prednisolone acetate, prednisolone valerate, prednicarbate, prednisone, triamcinolone acetate, triamcinolone diacetate and triamcinolone acetonide.

Preferably, the glucocorticoid is hydrocortisone or a salt thereof.

Preferably, the glucocorticoid is a hydrocortisone chosen from benzodrocortisone (hydrocortisone 17-benzoate), hydrocortamate (hydrocortisone 21-(diethylamino) acetate), hydrocortisone aceponate (hydrocortisone 21-acetate 17a-propionate or HCA), hydrocortisone acetate, bendazac, hydrocortisone buteprate hydrocortisone (hydrocortisone 17a-butyrate 21-propionate), hydrocortisone butyrate (hydrocortisone 17a-butyrate), hydrocortisone 21-butyrate, hydrocortisone cypionate (hydrocortisone cyclopentanepropionate), hydrocortisone hydrogen succinate, hydrocortisone phosphate, hydrocortisone sodium phosphate, hydrocortisone sodium succinate, hydrocortisone succinate (hydrocortisone hemisuccinate), hydrocortisone tebutate, hydrocortisone valerate, hydrocortisone xanthogenic acid and hydrocortisone probutate.

More preferably glucocorticoid is hydrocortisone diester. Even more preferably, the glucocorticoid according to the invention is hydrocortisone aceponate (HCA).

Hydrocortisone aceponate is biologically inactive prior to rapid hydrolysis to three esters (hydrocortisone propionate, cortisone aceponate, cortisone propionate) that are ultimately transformed into hydrocortisone and subsequent metabolites similar to substances of endogenous origin. The mechanism of action is binding to the cytoplasmic glucocorticoid receptor, translocation of the receptor to the nucleus, and complex to the nucleus with a resultant initiation transcription of genes encoding for any inflammatory mediators such a cytokines and lipocortins. The lipocortins inhibit phospholipase A2 hence blocking the release of arachidonic acid from membrane phospholipids and preventing synthesis of prostaglandins and leukotrienes. This provides potent local activity with minimal systemic circulation.

HCA provides reduction of pain and inflammation which leads to a quicker and more complete recovery from mastitis.

According to the present invention, the antibiotic (i) chosen from penicillin, its derivatives and its salts, and the glucocorticoid (ii) are active agents, i.e. pharmaceutical or veterinary compounds showing therapeutic effects. They are also named Active Pharmaceutical Ingredients (APIs).

### Pharmaceutically Acceptable Medium of the Composition of the Invention of the Invention

The composition of the invention comprises the APIs in a pharmaceutically acceptable medium (or carrier). Said composition is a pharmaceutical composition.

The compositions of the invention also contains the APIs, preferably each API (i.e. the antibiotic (i) chosen from penicillin, its derivatives and its salts, and the glucocorticoid (ii); and optionally a second antibiotic chosen from beta-lactam antibiotics) is formulated in a pharmaceutically acceptable medium (or carrier).

Said APIs are preferably formulated in a composition that includes a pharmaceutically acceptable liquid or solid carrier, or carriers, including a variety of diluents or excipients known to those of ordinary skill in the art.

A particularly preferred pharmaceutically acceptable carrier is methyl ether propylene glycol.

The composition may additionally include additives, excipients, thickeners and/or other substances which allow for a more effective administration.

Preferred excipients are those showing an antiseptic activity. They may be selected in the group comprising, but not limited to, alcohols (such as alcohol Benzoate, benzyl alcohol, ethanol), solvents (for example Propylene glycol, Glycerine), surfactants (such as non-ionic surfactants), emollients (such as Macrogol Cetostearyl Ether, Diisopropyl Adipate), dispersing agents (such as Sorbitan sesquioleate), flavoring agents (for example vanillin), Chelating agents (for example EDTA disodium salt), oils (for example vegetable oils, preferably olive oil, including Oleic Acid, linolenic and linoleic acids, coconut oil, Caprylic/Capric Triglycerides, Coco-Caprylate/Caprate), vegetal extracts which may be essential oils or not (such as turmeic oil, Melaleuca oil, calendula, Oregon grape, thyme, garlic, sage, lemon grass, peppermint, catnip, tea tree, hypericum, rosemary, citrus, eucalyptus, avies pectinata wood oils), additives (for example Nitric oxide, sodium Benzoate, Silicon dioxide, Titanium dioxide, tanic acid, Curcumin (turmeric)), Polygodial, thymol, Nisine, polyhexanide, chlorobutanol, hydrogen peroxide, benzethonium chloride, silver (such as nitrate, chlorure or colloidal), silver salts, iodine or iodine derivatives (such as Povidone (PVP) iodine), disinfecting agents (for example chlorhexidine, chloramine T, potassium permanganate), preservative agents (for example Bronopol, E-280 (propionic acid), E281 (sodium propionate), E282 (calcium propionate), E36 (formic acid), Phenoxyethanol, Thimerosal, potassium sorbate, Polylysine, citric acid, mix of citric (25%), sorbic (16.7%) acids, sodium Thiosulfate, sodium thiosulfate pentahydrate) or sweetening agent (for example honey).

In a preferred embodiment, excipients having an antiseptic activity are selected from Coco-Caprylate/Caprate, diisopropyl adipate, coconut oil, Nisine, PVP iodine, Alcohol Benzoate, Caprylic/Capric Triglycerides, Glycerine, olive oil (including Oleic Acid, linolenic and linoleic acids), Polylysine, Honey, Propylene glycol, Macrogol Cetostearyl Ether, nikkol so-15 VL Sorbitan sesquiolate and Silica dioxide.

In a more preferred embodiment, excipients having an antiseptic activity are PVP iodine, Coco-Caprylate/Caprate, diisopropyl adipate, coconut oil and Nisine.

Formulations particularly adapted to treat mastitis for non-human animals, preferably for lactating ruminants, also called intramammary infusion, are usually composed of a vehicle or carrier associated with an active ingredient. Intramammary formula can be in the form of a liquid product, a semi-solid or a thixotropic product such as a solution, a suspension, a paste, a gel or a cream formulation.

In a preferred embodiment the composition according to the invention comprises the APIs, as described above, which are solubilized in the pharmaceutically acceptable carrier. In another preferred embodiment, the APIs are in the form of particles in the intramammary formulations.

The intramammary composition of the invention may further comprise any suitable pharmaceutically acceptable ingredients which are known in the pharmaceutical art. They may be selected from oil, medium chain triglyceride, wax (e.g. microcrystalline or beeswax), fatty acid, fatty acid derivative, viscosity control agent or thickener (stearate salt, silicon dioxide, cellulose); pigment, opacifier, dispersing agent; emulsifier; stabilizer; surfactant, humectant; antioxidant; antibacterial agent, antifungal agent, preservative, emollient, polymers, any compound conferring organoleptic properties (flavor, dye, perfume, etc.), water and combinations thereof. The oil used is preferably selected from vegetable (e.g. natural plant oil) or mineral origin (e.g. paraffin, white petrolatum, yellow petrolatum). The fatty acid derivatives are either organic or inorganic compounds such as aluminium stearate, magnesium stearate, zinc stearate, stearic acid, hydrogenated vegetable oil and the like. Mineral particles such as, but not only, silica, clay, calcium carbonate are eventual excipients that can be used as thickener.

In a particular embodiment, the composition of the invention can be administered for treatment of mastitis by inserting the cannula nozzle of a mastitis syringe into the external orifice of the teat canal of an udder of a milk producing animal and infusing the at least one glucocorticoid and the at least one antibiotic into the udder.

Preferably, the glucocorticoid is present in the composition of the invention, in an amount comprised between 10 and 40 mg, preferably between 15 and 25 mg, more preferably of around 20 mg.

Preferably, HCA is present in the composition or the product of the invention, in an amount comprised between 10 and 40 mg, preferably between 15 and 25 mg, more preferably of around 20 mg.

Preferably, the antibiotic, preferably penicillin G procaine, is present in the composition of the invention in an amount comprised between 500 and 2000 mg, preferably between 700 and 1500 mg, more preferably of around 1000 mg.

Preferably, the amount of penicillin G alone in the composition of the invention is comprised between 500 and 1000 mg, preferably between 350 and 750 mg.

Preferably, the quantity of glucocorticoid to be applied in an udder quarter via the teat canal is comprised between 10 and 50 mg, preferentially between 15 and 25 mg, more preferably 20 mg. Such glucocorticoid may be administered at least once a day for 1 to 10 days; depending on the infection importance, the glucocorticoid may be administered for 1, 2, 3, 4, 5, 6, 7, 8, 9 or 10 days. Preferably, such glucocorticoid is administered once a day for 3 days; this corresponds to a full course of treatment.

According to the invention, as shown in the examples, 24 hourly intervals in treatment result in non-inferior cure rates compared to 12 hourly intervals when 3 doses are given to cows milked twice daily. Thus treatment once a day (24 hourly intervals), preferably for 3 days, is a preferred dosage regimen.

For example, a volume of 4 g of a pharmaceutical composition of the invention is administered in the udder via a teat canal and the quantity of said glucocorticoid in said pharmaceutical composition is preferably comprised between 10 and 50 mg, preferably between 15 and 25 mg, more preferably of around 20 mg.

Preferably, the quantity of antibiotic to be applied in an udder quarter via the teat canal is comprised between 500 and 2000 mg, preferably between 700 and 1500 mg, more preferably of around 1000 mg. Such antibiotic may be administered at least once a day for 1 to 10 days; depending on the infection importance, the antibiotic may be administered for 1, 2, 3, 4, 5, 6, 7, 8, 9 or 10 days. Preferably, such antibiotic is administered once a day for 3 days; this corresponds to a full course of treatment.

For example, a volume of 4 g of a pharmaceutical composition of the invention is administered in the udder via a teat canal and the quantity of said antibiotic in said pharmaceutical composition is preferably comprised between 500 and 2000 mg, preferably between 700 and 1500 mg, more preferably of around 1000 mg.

Preferably, the pharmaceutical composition for the treatment of mastitis of the invention is administered in the udder via the teat canal once per day, for 1 to 10 days, preferably for 1 to 5 days, and even more preferably for 3 days.

In a preferred embodiment, each teat of the udder of the infected lactating ruminant is treated with a pharmaceutical composition according to the invention.

Preferably, the pharmaceutical composition of the invention is formulated in a syringe.

Preferably, the pharmaceutical composition of the invention are infused into the teat canal of each infected udder quarter of the infected lactating ruminant immediately following milking. Preferably, the pharmaceutical compositions of the invention are administered three times at 24-hour intervals. If clinical resolution is not achieved following these three times, an extended course of up to 6 treatments may be given, preferably administered at 24-hour intervals.

Another aspect of the invention is a kit comprising:
a) a composition according to the invention,
b) a notice of use of the veterinary pharmaceutical product, and
c) an optional administration device.

In a preferred embodiment, the veterinary pharmaceutical product can be constituted by a first composition comprising, in a pharmaceutically acceptable medium, at least one antibiotic chosen from penicillin, its derivatives and its salts (such as benzylpenicillin (penicillin G) or penicillin G procaine) and, a second composition comprising, in a pharmaceutically acceptable medium at least one glucocorticoid (such as hydrocortisone aceponate (HCA)).

The first composition may comprise at least two antibiotics, wherein at least one antibiotic is chosen from penicillin, its derivatives and its salts, and at least one other is chosen from beta-lactam antibiotics.

Preferably, the kit according to the invention is used for treating mastitis.

Said composition in the kit according to the invention may be provided in a ready-to-use administration device or may be provided separately from the administration device.

Examples of administration device include, but are not limited to, pump spray, syringe or tube.

A particularly adapted administration device for the treatment of mastitis is a syringe.

The invention is now illustrated by the following examples.

The legends of the figures are as follows:

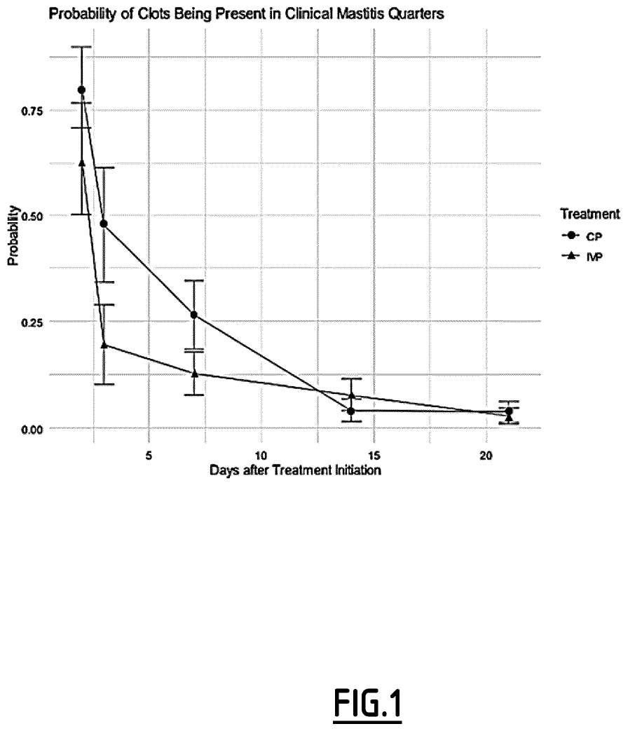

FIG. 1. Plot of predicted probability of clots being detected in milk from cows with clinical mastitis after treatment with penicillin (CP) or penicillin and HCA (IVP-combined treatment of the invention), over time from logistic regression with time by treatment interaction included. Time is treated as a factor variable. Error bars display the standard error of the probability estimates.

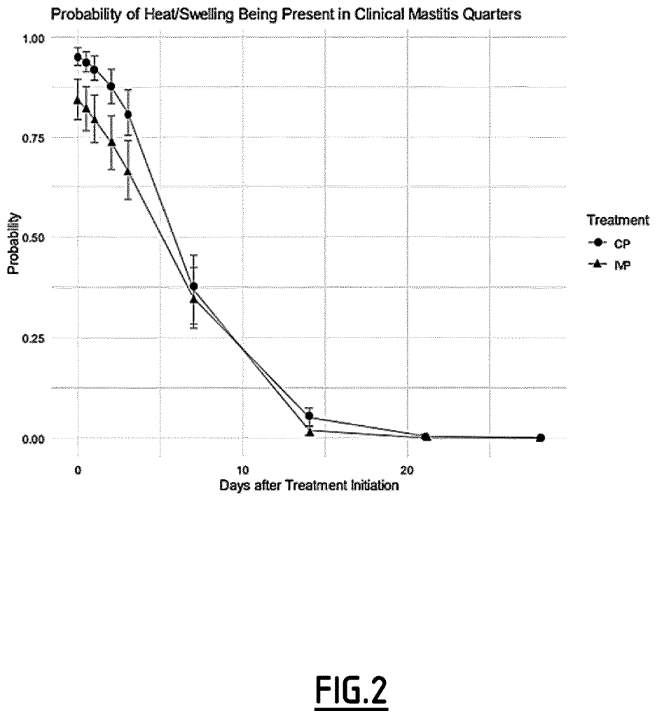

FIG. 2. Plot of predicted probability of heat/swelling being present in quarters with clinical mastitis after treatment with penicillin (CP) or penicillin and HCA (IVP-combined treatment of the invention), over time from logistic regression with time by treatment interaction included. Error bars display the standard error of the probability estimates.

### EXAMPLES

### Example 1: Assessing the Effects of Addition of HCA to Intramammary Antimicrobial for Therapy of Mastitis of Lactating Dairy Cows

### Summary

Intracillin Milking Cow Platinum is a new intramammary combining the antibiotic activity of penicillin with the anti-inflammatory effects of hydrocortisone aceponate (HCA). Each syringe of Intracillin Milking Cow Platinum contains 1000 mg penicillin G procaine and 20 mg HCA.

Intracillin Milking Cow Platinum is effective against Gram-positive mastitis organisms, including but not limited to:
Streptococcal species such as Streptococcus uberis,
Staphylococcal species including coagulase-negative Staphylococci and penicillin-sensitive Staphylococcus aureus.

Intracillin Milking Cow Platinum is also indicated for the reduction of the clinical signs of udder inflammation such as heat, swelling, clots and elevated somatic cell count (SCC) associated with mastitis.

The entire contents of one syringe should be infused into the teat canal of each infected quarter immediately following milking. Repeat the treatment twice more at 24-hour intervals. Three syringes provide a full course of treatment. If clinical resolution is not achieved following three doses, an extended course of up to 6 treatments may be given, administered at 24-hour intervals.

The recommended 24-hour treatment interval constitutes a change to current practice. The multi-arm, blinded, randomised, positively controlled intervention study, conducted using cows with naturally occurring mastitis, demonstrated 24 hour treatment intervals resulted in non-inferior cure rates compared to 12 hourly intervals when 3 doses were given to cows milked twice daily. In this study, cure rate was not depressed by 24 hour treatment intervals and twice a day milking which likely resulted in the removal of antibiotic via the milking process between doses. Herd owners may prefer that all treatments occur once daily to simplify management and minimise risks of incorrect treatment intervals occurring or the risk that withholding periods are not observed.

### Materials & Methods

This prospective, multi-arm, blinded, randomised, positively controlled intervention study was conducted using cows with naturally occurring clinical (n=61 cows in year 2), and subclinical (n=410 cows) mastitis, sourced from 20 farms (year 2) in the Waikato region of New Zealand. In addition, 59 cows from 6 farms enrolled in a similar study of clinical mastitis in the previous year (year 1) were included in the analysis of the clinical arm.

Whilst year 1 investigated cows milked twice a day, year 2 assessed cows being milked once a day for the clinical arm and twice a day for the sub-clinical arm.

### Test Items

IVP (“Investigational Veterinary Product”; combined treatment of the invention): 1000 mg penicillin G procaine and 20 mg HCA per syringe. The dose was one 4 g intramammary syringe per quarter.

CP (“Control Product”; penicillin G alone): 1000 mg penicillin G procaine per syringe. The dose was one 4 g intramammary syringe per quarter.

### Clinical Mastitis Arm

Cows diagnosed with clinical mastitis (i.e. presence of heat or swelling of the mammary gland, changes in milk composition such as clots or wateriness) were enrolled. These were assessed by technicians and then randomly assigned to be treated with 1000 mg of penicillin G procaine by intramammary infusion with and without HCA, at 24 hourly intervals on 3 occasions, with the cows milked once a day (year 2) or twice a day (year 1) during treatment period and for 5 subsequent days (i.e. the WHP). Treatment was allocated at cow level from a pre-prepared randomised treatment list.

Cows were examined at 7, 14 and 21 (+3) days post initiation of treatment to assess clinical signs (clot score, udder heat/swelling), and milk samples for microbiology (except day 28) were collected. In addition, an ~25 mL milk sample was collected for SCC determination at each time point. Clinical scores for mastitis were recorded at these time points and at 0.5, 1, 2 to 3 days post-treatment in year 1 of the clinical arm. The clinical arm in year 2 had an additional time point at 28 days where a SCC and clinical scores were obtained.

### Results

### Bacteriological Cure

Bacteriological cure was defined as having occurred where the 3 post-treatment milk samples (i.e. days 7, 14, and 21 post initiation of treatment) did not isolate the bacteria which had been present prior to initiation of treatment (Day 0). If the Day 0 sample was contaminated or was no growth, the bacteriological cure was coded as null.

### Clinical Arm

There was no difference in the proportion of quarters infected with different bacterial species prior to treatment between the treatment groups (p=0.5).

There was no difference (p=0.71) in the unadjusted cure rate for quarters diagnosed with clinical mastitis and treated with the IVP or CP.

TABLE 1
Raw cure rates for quarters diagnosed with clinical mastitis and
having a positive pre-enrolment culture result by treatment.

| Treatment | Cure Rate |
|---|---|
| CP | 61.2% (30/49) |
| IVP | 66.7% (36/54) |
| Total | 64.1% (66/103) |

There was no difference in the estimated marginal means of the cure rate for each treatment (p=0.87). The model also included study, age (2 vs 2+) and pre-enrolment pathogen group.

TABLE 2
Estimated marginal means, standard error (SE) and 95% confidence
intervals (CI) for the cure rate for treatment of quarters
diagnosed with clinical mastitis and treated with the IVP
(penicillin and HCA) or the CP (penicillin).

| Treatment | Cure Rate | SE | 2.5% CI | 97.5% CI |
|---|---|---|---|---|
| CP | 65.1 | 7.6 | 49.2 | 78.2 |
| IVP | 66.7 | 7.1 | 51.6 | 79.0 |

### Sub-Clinical Arm

The distribution of pathogens did not differ (p=0.55) between treatment groups following grouping of pathogens into “Minor” (Corynebacterium spp, and CNS), “Mixed” (2 pathogens were isolated from the same quarter), “Strep” (Strep. uberis and Strep. dysgalactiae), “SA” (Staph. aureus), and “Other” (all other pathogens).

There was no difference (p=0.41) in the raw proportional cure rates between treatments for subclinically infected quarters.

TABLE 3
Unadjusted percentage (number of quarters cured/total quarters
in group) cure rates, for quarters with subclinical mastitis treated
with either penicillin (CP) or penicillin and HCA (IVP).

| Treatment | Cured |
|---|---|
| CP | 64.4% (221/343) |
| IVP | 61.1% (204/334) |
| Total | 62.8% (425/677) |

Treatments did not differ in the estimated marginal means of the cure rate (p=0.77). The final model also included breed, quarter location, frequency of treatment and adjusting for clustering of glands within individual cows and herds.

TABLE 4
The estimated margin means (SE); 95% confidence intervals (CI)
for bacteriological cure for quarters with subclinical mastitis
and treated with penicillin (CP) or penicillin and HCA (IVP).

| Treatment | Cure Rate | SE | 2.5% CI | 97.5% CI |
|---|---|---|---|---|
| CP | 66.2% | 3.6% | 58.8% | 72.9% |
| IVP | 64.9% | 3.7% | 57.4% | 71.8% |

The bacteriological cure of subclinically infected quarters after treatment with penicillin and HCA was non-inferior to penicillin as the lower limit of the 95% confidence interval of the difference in estimated marginal means is less than 10% absolute cure rate.

The bacteriological cure rate of subclinical mastitis cases did not differ between quarters treated 3 times at 12 or 24 hour intervals (p=0.65). Treatment at 24 h intervals for 3 treatments was non-inferior to treatment at 12 h intervals for 3 treatments, as the lower limit of the 95% confidence interval of the difference in estimated marginal mean is less than 10% below the control cure rate.

TABLE 5
The estimated margin means (SE), 95% confidence intervals (CI)
for bacteriological cure of quarters with subclinical mastitis
treated at 12- or 24-hourly intervals with penicillin or penicillin
and HCA from a generalised linear model taking into account the
effect of breed, front vs rear quarter, treatment and adjusting
for clustering of gland within cow and cow within herd.

| Frequency | Cure Rate | SE | 2.5% CI | 97.5% CI |
|---|---|---|---|---|
| 12 | 64.6% | 3.6% | 57.2% | 71.4% |
| 24 | 66.5% | 3.6% | 59.1% | 73.3% |

### New Intramammary Infection by Treatment Group

### Clinical Arm

There was no difference between treatment groups in the proportion of quarters for which a new intramammary infection was detected: 13/62 (21.0%) vs 8/65 (12.3%) for quarters treated with penicillin vs penicillin and HCA, respectively (p=0.28). New infections were associated with Strep. uberis (n=3 quarters), Coagulase-Negative Staphs or CNS (n=6 quarters) and Corynebacterium species (n=1 quarter).

### Sub-Clinical Arm

There was no difference (p=0.62) between treatment groups in the proportion of quarters for which a new intramammary infection was detected. New infections were most commonly associated with CNS (n=24 quarters).

### Somatic Cell Count

### Clinical Arm

Treatment with the penicillin and HCA resulted in a lower overall log 10 SCC (3.02 (SE=0.07) vs 3.15 (SE=0.07)) for quarters treated with penicillin and HCA versus those treated with penicillin alone, respectively. Log 10 SCC varied across time (p<0.05) and there was a time by treatment interaction (p<0.05). Quarters in the penicillin and HCA group had a significantly lower log 10 SCC at Days 14 and 21, compared with those quarters treated with penicillin alone. The proportion of quarters within SCC>200,000 cells/mL over time following treatment did not differ between treatment groups (p=0.09).

TABLE 6
Estimated marginal means of log10 SCC with 95% confidence intervals for treatment with penicillin
alone (CP) or in combination with HCA (IVP) at each sampling time point. The difference between
the means (i.e. CP - IVP) and the p-value of this difference is also displayed.

| | | | | | | | Difference | |
|---|---|---|---|---|---|---|---|---|
| | CP | | | IVP | | | between | |
| Time | Marginal | CP 2.5% | CP 97.5% | Marginal | IVP 2.5% | IVP | treatment | |
| (days) | Mean | CI | CI | Mean | CI | 97.5% CI | means | p-value |
| 0 | 3.39 | 3.20 | 3.58 | 3.42 | 3.24 | 3.60 | −0.028 | 0.76 |
| 0.5 | 3.38 | 3.20 | 3.56 | 3.40 | 3.22 | 3.58 | −0.021 | 0.82 |
| 1 | 3.37 | 3.19 | 3.55 | 3.38 | 3.21 | 3.56 | −0.014 | 0.87 |
| 2 | 3.35 | 3.17 | 3.52 | 3.35 | 3.18 | 3.52 | −0.001 | 0.99 |
| 3 | 3.32 | 3.15 | 3.50 | 3.31 | 3.14 | 3.48 | 0.013 | 0.87 |
| 7 | 3.24 | 3.08 | 3.40 | 3.17 | 3.01 | 3.33 | 0.069 | 0.36 |
| 14 | 3.09 | 2.93 | 3.25 | 2.92 | 2.77 | 3.07 | 0.166 | 0.02 |
| 21 | 2.94 | 2.76 | 3.11 | 2.67 | 2.50 | 2.84 | 0.263 | 0.01 |
| 28 | 2.79 | 2.57 | 3.00 | 2.42 | 2.22 | 2.63 | 0.361 | 0.01 |

TABLE 7
Percentage of quarters diagnosed with clinical mastitis with a SCC
>200,000 cells/mL (number of quarters with SCC >200,000 cells/mL
over total in group) by days after treatment initiation having
been treated with penicillin (CP) or penicillin and HCA (IVP).

| | 0.5 | 1 | 2 | 3 | 7 | 14 | 21 | 28 |
|---|---|---|---|---|---|---|---|---|
| CP | 92.3% | 100% | 100% | 100% | 96.4% | 98.0% | 87.9% | 83.3% |
| | (12/13) | (20/20) | (25/25) | (31/31) | (54/56) | (48/49) | (43/49) | (20/24) |
| IVP | 100% | 100% | 100% | 100% | 90.2% | 76.8% | 71.1% | 71.4% |
| | (13/13) | (20/21) | (26/26) | (28/28) | (55/61) | (43/56) | (38/53) | (20/28) |

### Sub-Clinical Arm

There was no difference in post treatment quarter-level log 10 SCC between treatment groups (p=0.38). Time and front vs rear quarters were significant factors in the final model (p<0.001). There was no interaction of treatment with frequency of administration (p=0.36).

### Clot Scores

Treatment with the penicillin G procaine and HCA resulted in a trend towards a lower proportion of quarters without clots with an odds ratio of 0.51 (95% CI 0.0.22-1.15, p=0.1) relative to the positive control treated quarters (FIG. 1). Additionally, the clot score varied across time (p<0.001), but there was no significant time by treatment interaction (p=0.48). Marked clustering occurred within cow (Intraclass Correlation=0.41).

### Heat and Swelling Scores

Treatment with the penicillin G procaine and HCA reduced the heat or swelling score (p=0.01). Time was significant (p<0.001) and there was a treatment by time interaction (p=0.01). There was marked clustering within cow (Intraclass Correlation=0.44) (FIG. 2).

### Conclusions

This study demonstrates that addition of HCA to an intramammary formulation containing 1000 mg of penicillin G procaine resulted in similar bacteriological cure rates, but reduced local inflammatory responses in naturally acquired intramammary infections resulting in clinical mastitis, compared with treatment by infusion of 1000 mg of penicillin G procaine alone. The bacteriological cure rate of penicillin in combination with HCA was non-inferior to penicillin alone in quarters with naturally acquired subclinical intra-mammary infection. Additionally, it was concluded that 24 hourly intervals in treatment resulted in non-inferior cure rates compared to 12 hourly intervals when 3 doses are given to cows milked twice daily.

Frequency of administration did not affect microbiological cure and did not interact with treatment group. Penicillin is a time dependent antibiotic so that the pharmacokinetic parameter to maximise is the time above minimum inhibitory concentration as a percentage of treatment period. Antibiotic concentrations in the udder are expected to reduce at each milking (Whittern et al 2012), and then be elevated as another dose is administered. In this study, cure rate was not depressed by 24 hourly treatment intervals and twice a day milking which likely resulted in the removal of antibiotic via the milking process between doses. This may be related to the comparatively large dose (1000 mg) of penicillin G procaine administered, meaning that the concentration may not have fallen below the MIC for significant periods within the inter-dosing interval. The clinical significance of this finding is that 1000 mg penicillin G procaine intramammary products may be infused at 24 hourly intervals where twice a day milking is occurring. For managerial reasons herd owners may prefer that all treatments occur once daily to minimise risks of incorrect treatment intervals occurring or the risk that withholding periods are not observed.

Inclusion of HCA was associated with a reduction in scores associated with inflammation. For clinical cases HCA treatment reduced quarter-level SCC at 14, 21 and 28 days post-initiation of treatment, but not at earlier time points in the treatment course (Tables 6 and 7). Overall addition of HCA tended to reduce the clot scores, with numerically lower clot scores within the 1st week post treatment. HCA significantly reduced the heat/swelling scores in the 1st 3 days post-treatment. No clots were observed at 28 days post-treatment and only one quarter in each treatment group was observed with heat or swelling at Day 28. It should be noted that a number of cows were diagnosed with recurrent clinical mastitis and treated prior to Day 28; thus these animals are removed from the analyses of clinical scores. It should also be noted that while few animals had evidence of clinical mastitis by 3 weeks post treatment, the majority of quarters still had elevated (i.e. >200,000 cell/mL) SCC at Day 21 and Day 28 post-treatment. Given that inflammation occurs at both a local and systemic level, it is unsurprising to see a high level of clustering for clot and heat/swelling scores within a cow (i.e. if one quarter has heat/swelling then the other quarter(s) within the cow are more likely to also have heat/swelling). This may explain the marked difference in predicted prevalence from the model and the observed proportions in the raw data.

### Example 2: Residue Study

### Materials & Methods

### Test Item

1000 mg penicillin G procaine and 20 mg HCA per syringe.

The dose was one 4 g intramammary syringe per quarter.

### Study Design

Four groups of eleven cows (9+2 spares) were enrolled in the study.

Cows in Group 1 were milked once-a-day, and treated with the test item immediately following milking on study days −5, −4, −3, −2, −1 and 0 at a rate of one syringe per quarter. Cows in Group 2 were milked twice-a-day, and treated with the test item immediately following the AM milking on study days −5, −4, −3, −2, −1 and 0 at a rate of one syringe per quarter.

Cows in Group 3 were milked once-a-day, and treated with the test item immediately following milking on study days −2, −1 and 0 at a rate of one syringe per quarter. Cows in Group 4 were milked twice-a-day, and treated with the test item immediately following the AM milking on study days −2, −1 and 0 at a rate of one syringe per quarter.

Milk yields at the AM milking were recorded and milk specimens collected on days 4, 5 and 6 (Groups 1 and 3), or days 3, 4 and 5 (Groups 2 and 4). Penicillin concentrations were determined in the milk specimens. The limit of quantification (LoQ) was 0.00044 mg/kg and the limit of detection (LoD) was 0.00023 mg/kg. The maximum residue limit (MRL) was 0.004 mg/kg.

### Data Analysis

For analysis, observations below the LoQ were replaced with half the LoQ (0.00022 mg/kg), and observations below the LoD were replaced with half the LoD (0.00012 mg/kg).

Means (weighted by the milk yield recorded for each animal) and standard deviations for each time were calculated using Microsoft Excel. The upper conformance limit (UCL) for each time point were calculated in accordance with 39 ACVM 03/03 guidelines, i.e.:

UCL=weighted mean±4.143*SD/sqrt (number of observations).

### Results

TABLE 8
Summary statistics for study animals.

| | | | | No. | No. |
|---|---|---|---|---|---|
| | Weighted | | | animals | animals |
| | mean | | | below | below |
| Milking | concentration | SD | UCL | MRL | LoQ |
| Group 1 | | | | | |
| Day 4 | 0.00096 | 0.00212 | 0.00388 | 8 | 4 |
| Day 5 | 0.00011 | 0.00004 | 0.00018 | 9 | 9 |
| Day 6 | 0.00010 | 0 | 0.00010 | 9 | 9 |
| Group 2 | | | | | |
| Day 3 | 0.00210 | 0.00203 | 0.00491 | 7 | 0 |
| Day 4 | 0.00021 | 0.00024 | 0.00054 | 9 | 7 |
| Day 5 | 0.00009 | 0 | 0.00009 | 9 | 9 |
| Group 3 | | | | | |
| Day 4 | 0.00104 | 0.00155 | 0.00318 | 8 | 3 |
| Day 5 | 0.00016 | 0.00020 | 0.00044 | 9 | 8 |
| Day 6 | 0.00010 | 0 | 0.00010 | 9 | 9 |
| Group 4 | | | | | |
| Day 3 | 0.00177 | 0.00167 | 0.00408 | 7 | 1 |
| Day 4 | 0.00013 | 0.00004 | 0.00019 | 9 | 9 |
| Day 5 | 0.00010 | 0 | 0.00010 | 9 | 9 |

Data less than LoQ replaced by half the LoQ (0.00022 mg/kg) for calculation of mean and SD

Plotting the UCLs against time, the fit was improved by log-transforming the y-axis (from R² 0.76 to R² 0.86 for Group 1; from R² 0.82 to R² 0.99 for Group 2; from R² 0.83 to R² 0.99 for Group 3; from R² 0.77 to R² 0.87 for Group 4).
For Group 1, the UCL drops below the MRL 90 hours after the last treatment.
For Group 2, the UCL drops below the MRL 74 hours after the last treatment.
For Group 3, the UCL drops below the MRL 92 hours after the last treatment.
For Group 4, the UCL drops below the MRL 67 hours after the last treatment.

Based on these results, the WHP may be as follows:
Milk: Twice a day milking:
   For 3 treatments at 24 hour intervals-72 hours (6 milkings) (Group 4) For 6 treatments at 24 hour intervals-84 hours (7 milkings) (Group 2)
Milk: Once a day milking:
   For 3 or 6 treatments at 24 hour intervals-96 hours (4 once a day milkings) (Group 1&3)
Meat: 10 days.

As a comparison, the equivalent WHP label claims for Intracillin 1000 Milking Cow (1000 mg penicillin G procaine only) are:
Milk: Twice a day milking-96 hours (8 milkings).
Milk: Once a day milking-120 hours (5 once-a-day milkings).
Meat: 10 days.

The WHP for the combined treatment of the invention are therefore 24 hours shorter than for Intracillin 1000 Milking Cow (1000 mg penicillin G procaine only) for equivalent 3 syringe treatments for both twice a day and once a day milking.

### SUMMARY

This study demonstrates that penicillin residues from the use of the combined treatment of the invention are below MRL by 72 hours after the last treatment in cows milked twice daily and treated 3 times, 84 hours in cows milked twice daily and treated 6 times, and 96 hours in cows milked once daily and treated either 3 or 6 times.

These times are much shorter (at least 24 h) than for the equivalent regimes using Intracillin 1000 Milking Cow (1000 mg penicillin G procaine only).

This suggests that the addition of HCA has the effect of enhancing the removal of penicillin from the udder, as well as the substantial effects on inflammation seen in the clinical trials.

1-15. (canceled)

16. A method for the treatment or the prophylaxis of bacterial infectious diseases in infected lactating ruminants, comprising administering to said infected lactating ruminants a composition comprising, in a pharmaceutically acceptable medium, (i) at least one antibiotic chosen from penicillin, its derivatives and its salts, and (ii) at least one glucocorticoid, wherein penicillin and its derivatives are chosen from compounds of formula (I): 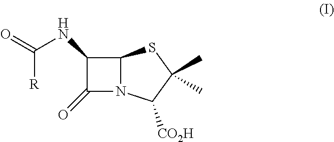
wherein R is an aralkyl, a phenoxymethyl radical, an alkyl radical having 1 to 10 carbon atoms or an alkenyl radical having 2 to 10 carbon atoms,
wherein said composition does not comprise any zirconium containing compound.

17. Method according to claim 16, which is for the reduction of the clinical signs of udder inflammation such as heat, swelling, clots and elevated somatic cell count associated with mastitis.

18. Method according to claim 16, wherein the withholding period of the antibiotic in the milk of said lactating ruminant is reduced compared to the withholding period of the antibiotic in the milk when said antibiotic is administrated alone.

19. Method according to claim 16, wherein the at least one antibiotic chosen from penicillin, its derivatives and its salts is penicillin G and more preferably penicillin G procaine, and the at least one glucocorticoid is hydrocortisone aceponate, wherein penicillin and its derivatives are chosen from compounds of formula (I): 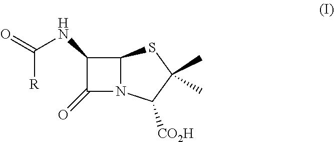
wherein R is an aralkyl, a phenoxymethyl radical, an alkyl radical having 1 to 10 carbon atoms or an alkenyl radical having 2 to 10 carbon atoms.

20. Method according to claim 16, wherein the antibiotic is present in the composition, in an amount comprised between 500 and 2000 mg, preferably between 700 and 1500 mg, more preferably of around 1000 mg.

21. A method for enhancing the removal of an antibiotic chosen from penicillin, its derivatives and its salts, in an infected lactating ruminant treated by said antibiotic, comprising administering to said infected lactating ruminant a composition comprising, in a pharmaceutically acceptable medium, hydrocortisone aceponate, wherein enhancing the removal of an antibiotic chosen from penicillin, its derivatives and its salts means that the antibiotic residue in an infected lactating ruminant treated by said antibiotic and by hydrocortisone aceponate is below maximum residue limit within at least 6 h, wherein penicillin and its derivatives are chosen from compounds of formula (I): 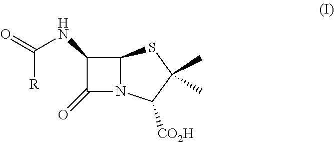
wherein R is an aralkyl, a phenoxymethyl radical, an alkyl radical having 1 to 10 carbon atoms or an alkenyl radical having 2 to 10 carbon atoms.

22. A Method for lowering somatic cell count in the milk of a lactating ruminant infected with a mastitis-causing organism, which comprises administering to said infected lactating ruminant a composition comprising, in a pharmaceutically acceptable medium, (i) at least one antibiotic chosen from penicillin, its derivatives and its salts, and (ii) at least one glucocorticoid, wherein lowering somatic cell count (SCC) in the milk means lowering SCC as compared to the SCC in the milk when said antibiotic is administrated alone i.e. without any glucocorticoid to the infected lactating ruminant, wherein penicillin and its derivatives are chosen from compounds of formula (I): 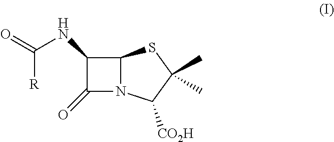
wherein R is an aralkyl, a phenoxymethyl radical, an alkyl radical having 1 to 10 carbon atoms or an alkenyl radical having 2 to 10 carbon atoms.

23. Method according to claim 16, wherein the bacterial infectious disease is mastitis.

24. Method according to claim 16, wherein the bacterial infectious disease is due to Gram-positive bacteria.

25. Method according to claim 24, wherein the Gram-positive bacteria are chosen from:
Streptococcal species such as Streptococcus uberis, and
Staphylococcal species including coagulase-negative Staphylococci, such as Staphylococcus xylosus, Staphylococcus sciuri and/or Staphylococcus saprophyticus, or penicillin-sensitive Staphylococcus aureus.
